(19) United States
(12) Patent Application Publication
(10) Pub. No.: US 2006/0142861 A1
(43) Pub. Date: Jun. 29, 2006

(54) Spinal disc replacement

(76) Inventor: Ian P. Murray, Phoenix, MD (US)

Correspondence Address: ARMSTRONG, KRATZ, QUINTOS, HANSON & BROOKS, LLP;Intellectual Property Law Offices, Suite 220, 502 Washington Avenue, Towson, MD, 21204, US

(21) Appl. No.: 11/025,741

(22) Filed: Dec. 29, 2004

(51) Int. Cl. A61F 2/44

(52) U.S. Cl. 623/17.13

(57) ABSTRACT

A spinal disc replacement has at least one bellows disposed between two spaced-apart plates. The bellows are compressible anteriorly/posteriorly and left/right. Several embodiments have top plates and bottom plates which are torsionally rotatable. Methods of use are provided.

### FIELD OF THE INVENTION

The present invention relates to a spinal disc replacement and, more particularly, to a bellows type device which flexes in all directions to accommodate natural bodily movements.

### BACKGROUND OF THE INVENTION

Spine non-fusion technologies have been under development for over 50 years. Many designs over the years have been purely theoretical. Consequently, many of the designs, patents and ideas were abandoned well before any clinical work was performed. In the past two years, non-fusion technology and philosophy has evolved from the clinical setting to a realized industry.

Total disc replacements are presently being sold worldwide. The rapid growth of the market may be attributed to:
Rapid uptake of total disc replacements through comprehensive surgeon training and education.
Steady sales growth and continued increases of annual usage.
Surgical Technique training workshops.
Philosophical education on the biomechanics, theoretical advantages, and presentation of long-term clinical data on various technologies.
Better understanding of the science of non-fusion and the associated segmental benefits of preserving motion and maintaining form and function.
Improved easy-to-use instrumentation that makes the procedure repeatable and reproducible.

The relief of low back and leg pain is the primary endpoint to spine surgery. One of the most integral components to the relief of pain and to ensuring success is the surgical technique and implant design. The currently offered Total Disc design technologies are anterior surgical procedures. In U.S. Pat. No. 4,932,975, Main et al disclose a vertebral prosthesis including a pair of housings having side and end walls forming a chamber in each housing. A suspension plane is in each housing, and at elastometric medium surrounds each suspension plate. The two suspension plates are interconnected by an expandable bellows.

Fuhrmann et al in U.S. Pat. No. 5,002,576 disclose an intervertebral disk endoprosthesis filled with a viscoelastic material, and a circular or elliptical corrugated tube surrounds the viscoelastic material and is contiguous thereto.

Fleischmann et al in U.S. Pat. No. 6,375,682 disclose a spinal prosthesis device which is expanded (or retracted) hydraulically.

In U.S. Pat. No. 6,395,032, Gauchet discloses an intervertebral disc prosthesis having a liquid in a closed chamber formed by a flexible seal between two opposing plates. A body, disposed in the chamber, has a plurality of pores at an outer surface thereof, and further has cavities extending from the pores in the body. The materials of the body and the liquid, respectively, are selected such that a compressive force must be applied to the liquid to force it into the cavities.

In U.S. Pat. No. 6,527,804 Gauchet et al disclose an intervertebral disc prosthesis having a body providing a cushion between two plates, and further having a compressible fluid (such as a gas) extending around the periphery of the body. The cushion exhibits a hysteresis-shaped curve of mechanical reaction to a compression as a function of a variation in a dimension of the cushion in the direction of the compression.

Gauchet also discloses, in U.S. Pat. No. 6,582,466, a prosthesis in which a compressible fluid in a sealed chamber is provided with a compressible body having a resistance to compression forces which is greater than that of the fluid.

Gauchet further discloses, in U.S. Pat. No. 6,582,468, a prosthesis having a compressible body in a closed fluid chamber formed by a flexible seal extending between a pair of opposed plates. The flexible seal enables the plates to move relative to each other.

In U.S. Patent Published Application No. 2003/0009223, Felding et al disclose a prosthesis having a caudal disc which is parallel to, and axially spaced from, a cranial disc. A spring means is disposed between the discs to elastically support the discs upon compression. the material of the spring means is a memory-metal alloy which has super elastic properties at body temperature.

In U.S. Patent Published Application No. 2003/083719, Kuslich et al disclose a device for connecting and stabilizing a spinal deformity. At least two stacked rods, closely spaced and parallel to each other, are connected to bone anchors for compressing the rods tightly together.

Ferree et al, in U.S. Patent Published Application No. 2003/0074076, disclose an artificial disc replacement (ADR) which includes a pair of end plates, a cushioning component therebetween, and a filler material within the cushioning component.

However, despite all of the interest and activity in the field, there is a need for an improved replacement device. There is specially a need for a device which accommodates normal bodily movement and which can be used with less invasive posterior surgical techniques.

### BRIEF SUMMARY OF THE INVENTION

It is an object of the present invention to provide a spinal disc replacement for damaged regions of the spine to avoid spinal fusion type surgery.

It is further an object of the present invention to provide a device having at least one bellows to be surgically implanted in the spine to replace a damaged spinal disc.

It is another object of the present invention to provide a device which compressible anteriorly/posteriorly and which is compressible medially/laterally.

It is still another object of the invention to provide a device which has rotational torsion between a top plate and a bottom plate.

In accordance with the teachings of the present invention there is disclosed a spinal disc replacement adapted to be surgically implanted in the vertebrae of a human patient. A device has at least one bellows disposed between a pair of spaced-apart top and bottom plates secured together peripherally of the bellows for retaining the bellows therebetween. The bellows is encapsulated to form a barrier against bodily fluids contacting the bellows. The bellows accommodates normal bodily movements of a patient receiving the spinal disc replacement, including twisting movements of the spinal disc replacement to a limited angular degree and in either direction circumferentially about a central longitudinal axis of the bellows.

In further accordance with the teachings of the present invention, there is disclosed a bellows assembly to be inserted into a patient's spinal column for replacement of a ruptured disc in lieu of a fusion procedure. The device has at least one bellows welded to top and bottom plates, respectively. The bellows and each of the plates are formed from different metallic materials, and an insulating member is disposed between the bellows and each of the plates, thereby preventing an electrolytic action between the bellows and the plates, respectively.

In still further accordance with the teachings of the present invention, there is described a bellows assembly to be inserted into a patient's spinal column for replacement of a ruptured disc in lieu of a fusion procedure. The assembly has at least one bellows welded to top and bottom plates, respectively, wherein the assembly can flex in a compressive mode substantially along the longitudinal axis of the bellows and between the respective plates. The assembly can accommodate pivotal movement of the assembly in a torsional mode and, to a limited degree, circumferentially about the longitudinal axis of the bellows.

Additionally, in accordance with the teachings of the present invention there is disclosed both an anterior and a posterior method of replacing the damaged spinal disc.

These and other objects of the present invention will become apparent from a reading of the following specification taken in conjunction with the enclosed drawings.

### BRIEF DESCRIPTION OF THE DRAWINGS

FIG. 1 is a perspective anterior view of a human spine having the device of the present invention surgically implanted therein.

FIG. 2 is a perspective view of the device of the present invention.

FIG. 3 is a top view of the device of the present invention.

FIG. 4 is a partial cross-section view of the present invention.

FIG. 5 is a perspective view of a bellows.

FIG. 6 is an exploded side view of a preferred embodiment of the present invention.

FIG. 7 is an end view showing anterior/posterior compression.

FIG. 8 is a side view showing left/right compression.

FIG. 9 is a top view showing torsional rotation between the top plate and the bottom plate.

FIG. 10 is a perspective view of the device showing torsional rotation of the plates in opposite directions.

FIG. 11 is a perspective view of the device showing torsional rotation of the plates in the same direction.

FIG. 12 is a perspective view of the device showing torsional movement of only the top plate.

FIG. 13 is a perspective view of the device showing torsional movement of only the bottom plate.

FIG. 14 is an exploded view of the device having a dome on the housing plug.

FIG. 15 is an exploded view of the two bellows device showing the bellows connected directly to the top plate and the bottom plate.

FIG. 16 is an exploded view of the device having one bellows connected directly to the top and bottom plates.

FIG. 17 is a bottom view of the top plate of the device of FIG. 16 shown for the right or left insertion.

FIG. 18 is an exploded view of the device having one bellows similar to FIG. 14.

FIG. 19 is a bottom view of the top plate of the device of FIG. 18 shown for right or left insertion.

FIG. 20 is a partial cut-away view of the device having a single bellows and insulator panels above and below the bellows.

FIG. 21 is a bottom view of the top plate of the device shown for right or left insertion.

FIG. 22 is a perspective view of the surgical procedure being performed posteriorly.

### DETAILED DESCRIPTION OF THE INVENTION

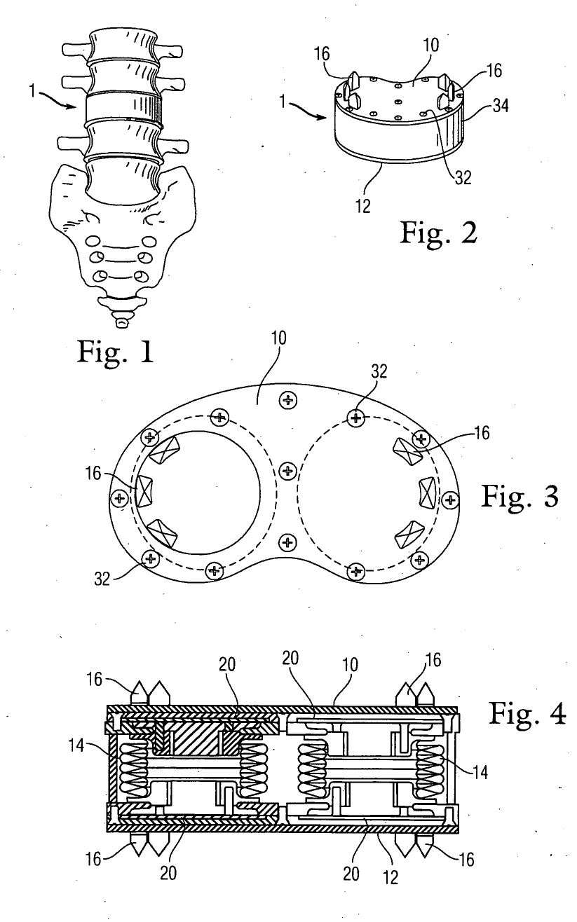

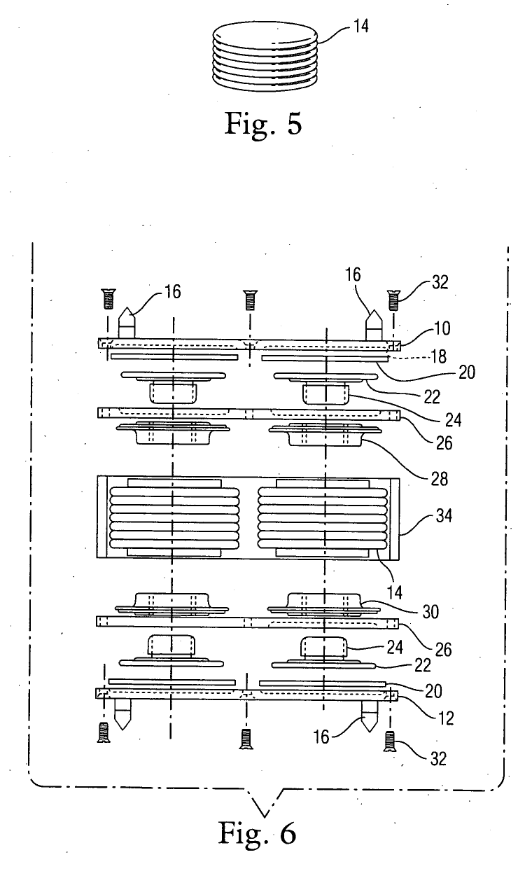

Referring now to FIGS. 1-5, the device of the present invention has a top plate 10 and a spaced-apart bottom plate 12. At least one bellows 14 is disposed between the top plate 10 and the bottom plate 12. In a preferred embodiment, the top plate 10 and the bottom plate 12 are kidney shaped and two bellows 14 are disposed side-by-side between the plates.

FIG. 6 shows the components of a preferred embodiment of the present invention. The top plate 10 is formed with a plurality of spikes 16 formed on an upper surface. The spikes 16 engage the vertebrae of the spine immediately above the device when implanted in the patient. The lower surface of the top plate 10 has a well 18 formed therein. An insulator panel 20 formed from a bearing material such as polyacetal or UHMWPE is received in the well 18. Other materials known to persons skilled in the art may be used. This insulator panel separates the bellows 14 from the top plate 10 and prevents the formation of an electrolitic reaction due to the presence of dissimilar metals. The top and bottom plates may be made from metal which are not the same as the bellows. If an electrolitic reaction were to occur, the device could be damaged by corrosion and failure could occur.

Abutting the insulator panel 20 is a housing plug 22 which has a depending cylinder 24 of a smaller diameter than the housing plug 22. The top of the housing plug 22 is disposed in an opening in a locator plate 26 with the depending cylinder 24 extending through the opening in the locator plate 26. The depending cylinder 24 is disposed in a top housing 28. The top housing 28 serves as a top end of the bellows 14.

The bellows 14 is a plurality of vertically joined rings forming a unitary bellows. The rings may be of the same or of different thickness to obtain a desired modulus of elasticity. The bellows may also be coated with a polymer if desired.

The bottom of the device is the reverse of the top, having, in sequence, a bottom housing 30, a locator plate 26, a housing plug 12, with a cylinder 24 formed thereon, an insulator panel 20 and a bottom plate 12 with exterior spikes 16. The device is secured with a plurality of fasteners 32, the top plate 10 and the bottom plate 12. Preferably, the respective locator plates 26 are welded to the top plate 10 and the bottom plate 12. An insulator barrier 34, preferably of an elastometric material, encapsulates the outer periphery of the device to keep out body fluids.

The above description addresses only one bellows but as noted above, two bellows may be disposed between the top plate 10 and the bottom plate 12. The second bellows is mounted and disposed in an identical manner to the first bellows.

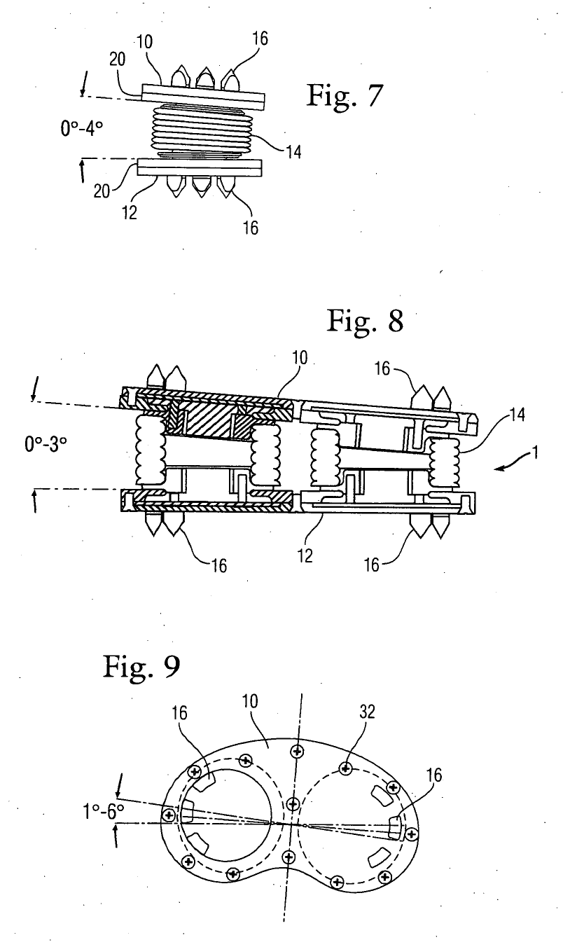

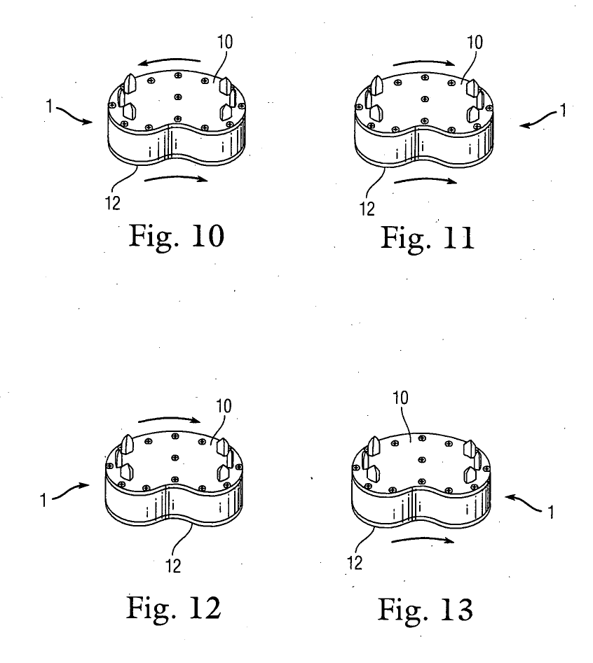

The device 10 is compressible anteriorly/posteriorly from approximately 0°-4° as shown in FIG. 7. As shown in FIG. 8, the device is compressible approximately 0°-3° left/right. The assembly flexes in the compressive modes substantially along a longitudinal axis of the bellows. Because of the low coefficient of the friction of the material from which the insulator panel 20 is formed, the top plate 10 and the bottom plate 12 may rotate torsionally approximately 1°-6°. Each plate may rotate independently of the other plate or both may rotate simultaneously (FIGS. 9-13). The multi-dimensional movement of the device provides movement of the vertebrae of the surgically-corrected spine which simulates movement of a person with a healthy spine.

The device does not require any fluid to be contained within the bellows.

The device may be made without the insulator. As shown in FIG. 14, the housing plug 22 is formed with an arch or dome 36 on the top surface. The dome contacts the inside of the well 18 in the top plate 10. The bottom of the bellows 14 is received directly in the well in the bottom plate 12. The device with the dome top housing plug also is compressible approximately 0°-4° anteriorly/posteriorly and approximately 0°-3° left/right. Due to the domed surface, the device also has transitional rotation of approximately 1°-6° between the top plate and the bottom plate circumferentially about the longitudinal axis of the bellows.

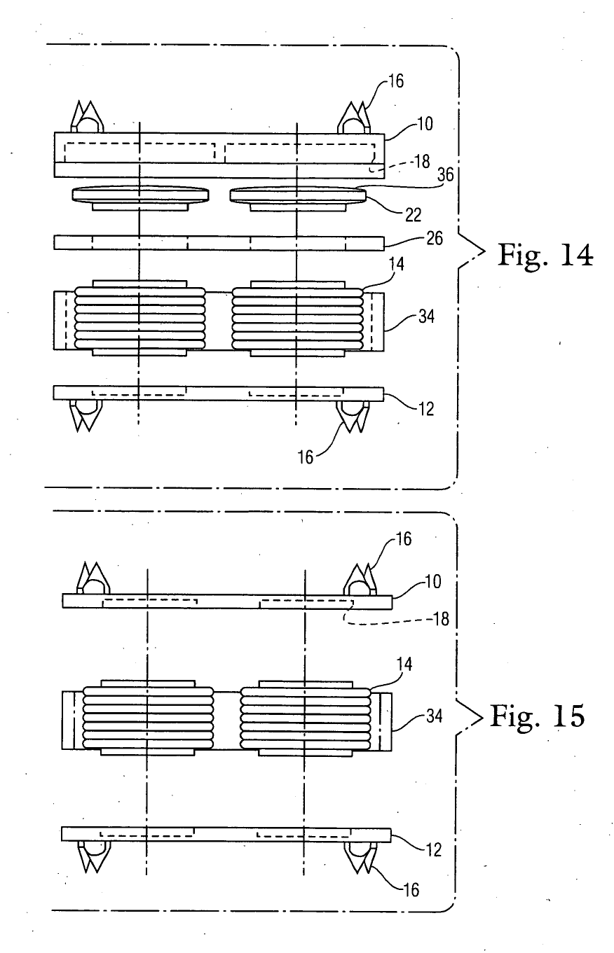

The device may also be made without a housing plug, an insulator, a housing or a locator plate. As shown in FIG. 15, the bellows 14 are directly connected to the respective top plate 10 and lower plate 12. This device is compressible approximately 0°-4° anteriorly/posteriorly and approximately 0°-3° left/right. However, the device has no translational rotation properties.

The device with the kidney shaped plates and two bellows is surgically implanted in the patient by anterior procedures which require an incision abdominally in the patient.

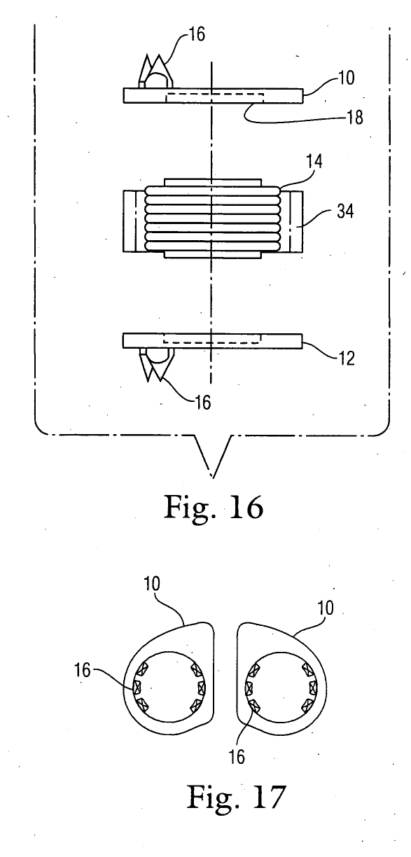

A device formed having only one bellows may also be made. As shown in FIG. 16, the device is essentially one-half of the device in FIG. 15. There is only a bellows 14 connected directly to the top plate 10 and the bottom plate 12. The device is formed as being for the right side or the left side of the spine as shown in FIG. 17.

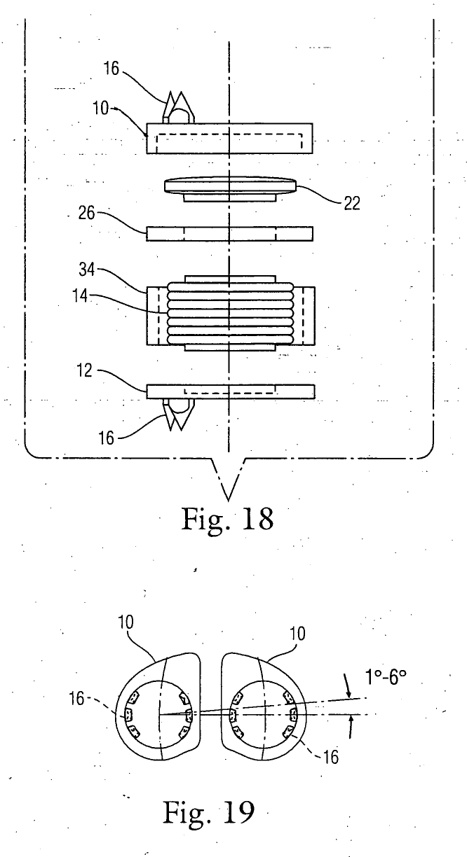

Similarly, as shown in FIGS. 18-19, a single bellows device similar to the device of FIG. 14 may be made. The device has the dome 36 on the housing plug 22. It also is formed for the right side or the left side of the spine. The device has torsional rotation of approximately 1°-6° of the top plate. The device is compressible approximately 0°-4° anteriorly/posteriorly and is compressible approximately 0°-3° left/right as the device is described above.

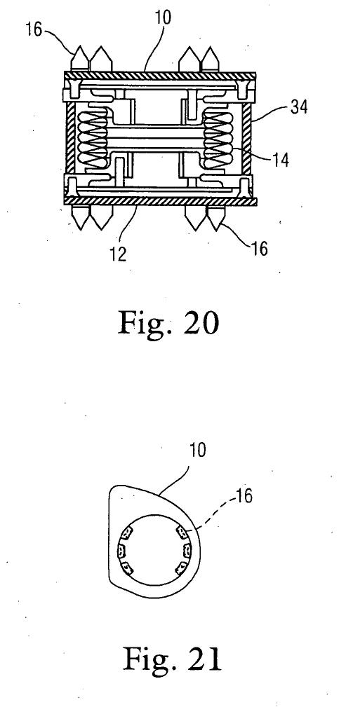

Also, a single bellows device having the insulator panel 20 may be made as shown in FIGS. 20-21. The device is formed for the right side or the left side of the spine. It has the torsional rotation of approximately 1°-6° of the top plate and the bottom plate. The device is compressible approximately 0°-4° anteriorly/posteriorly and is compressible approximately 0°-3° left/right as the device described above and shown, as a double bellows device in FIGS. 3-6.

The device is surgically implanted in the spine of a human being by making an incision in the abdominal area of the patient and providing access to the spine. The damaged region of the spine is removed. The device having at least one bellows and preferably having a kidney shaped top and bottom plate is inserted into the spine where the damaged region had been removed.

Techniques to be used by the surgeon will be developed and refined.

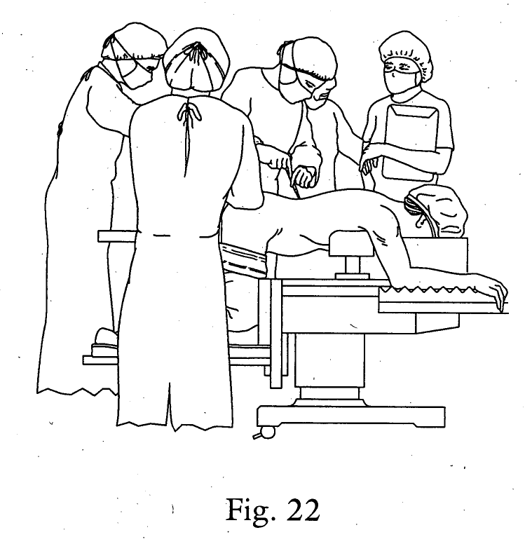

The device may also be implanted in the spine of a human being by making a midline incision posteriorly (FIG. 22). The damaged region of the spine is removed. A device having a single bellows and configured to fit the right side of the spine is inserted into the spine where the damaged region had been removed. A device having a single bellows and configured to fit the left side of the spine is inserted where the damaged region had been removed. The two inserted devices are adjacent to one another.

The type of device is selected by the surgeon depending upon the needs of the patient. The device may be any of those described above, i.e., the device without a domed housing plug, the device with the domed housing plug or the device with insulator panel.

The bellows 19 preferably is made from stainless steel and the top and bottom plate are formed form implantable grade titanium alloy or other alloy suitable to the practice.

The devices may be made in small, medium or large sizes. Under maximum anatomic material load of 1200 N±10 bellows compression distance is 9.5 mm to 6.5 mm.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A spinal disc replacement adapted to be surgically implanted in the vertebrae of a human patient, comprising at least one bellows disposed between a pair of spaced-apart top and bottom plates secured together peripherally of the bellows for retaining the bellows therebetween, the bellows being encapsulated to form a barrier against bodily fluids contacting the bellows, and the bellows accommodating normal bodily movements of a patient receiving the spinal disc replacement, including twisting movements of the spinal disc replacement to a limited angular degree and in either direction circumferentially about a central longitudinal axis of the bellows.

2. The spinal disc replacement of claim 1, wherein two bellows are disposed between the pair of spaced-apart top and bottom plates, each bellows having a top end and a bottom end, the respective top end and bottom ends being connected to the spaced-apart plates, such that the bellows are side-by-side and are compressible between the spaced-apart plates.

3. The spinal disc replacement of claim 2, wherein the plates have approximately 0°-4° of anterior/posterior compression.

4. The spinal disc replacement of claim 2, wherein the plates have approximately 0°-3° of left or right compression.

5. The spinal disc replacement of claim 1, wherein the plates have a torsional rotation of approximately 1°-6° with respect to one another.

6. The spinal disc replacement of claim 1, further comprises a respective bearing surface disposed between the ends, the bellows and the top and bottom plate.

7. The spinal disc replacement of claim 1, wherein the bellows are encapsulated within a insulator barrier, the barrier extending between the top plate and the bottom plate.

8. The spinal disc replacement of claim 1, wherein the top plate and the bottom plate each have a porous surface to facilitate fusion with the vertebrae.

9. The spinal disc replacement of claim 1, wherein the at least one bellows is formed of a material having a selected modulus of expansion.

10. the spinal disc replacement of claim 1, wherein the at least one bellows is formed form a plurality of rings, each ring having a selected thickness.

11. A bellows assembly to be inserted into a patient's spinal column for replacement of a ruptured disc in lieu of a fusion procedure, comprising at least one bellows welded to top and bottom plates, respectively, the bellows and each of the plates being formed from different metallic materials, and an insulating member between the bellows and each of the plates, thereby preventing an electrolytic action between the bellows and the plates, respectively.

12. The bellows assembly of claim 11, wherein the assembly is completely encapsulated peripherally thereof, thereby precluding bodily fluids from affecting the bellows assembly.

13. The bellows assembly of claim 11, wherein the assembly can flex in a compressive mode substantially along the longitudinal axis of the bellows and between the respective end plates, yet accommodate pivotal movement of the assembly in a torsional mode and to a limited degree circumferentially about the longitudinal axis of the bellows.

14. A bellows assembly to be inserted into a patient's spinal column for replacement of a ruptured disc in lieu of a fusion procedure, comprising at least one bellows welded to top and bottom plates, respectively, wherein the assembly can flex in a compressive mode substantially along the longitudinal axis of the bellows and between the respective plates, yet accommodate pivotal movement of the assembly in a torsional mode and to a limited degree circumferentially about the longitudinal axis of the bellows.

15. A method of surgically implanting a spinal disc replacement in a damaged region of the spine of a human being patient comprising the steps of:
providing a device having a top plate and a spaced-apart bottom plate, at least one bellows disposed between and connected to the plates,
making an incision in the abdominal area of the patient and providing access to the spine,
removing the damaged region of the spine,
inserting the device having the at least one bellows therein into the spine where the damaged region had been removed.

16. A method of surgically implanting a spinal disc replacement in the spine of a human being patient comprising the steps of:
providing two devices, each device having a top plate and a bottom plate, a bellows disposed between, and connected to, the plates,
making an incision posteriorly in the patient to provide access to the spine,
removing the damaged region of the spine,
inserting one of the devices into the right side of the spine where the damaged region had been removed,
inserting the other of the devices into the left side of the spine where the damaged region had been removed, the two devices being adjacent to one another.
